(19) United States
(12) Patent Application Publication
(10) Pub. No.: US 2010/0310511 A1
(43) Pub. Date: Dec. 9, 2010

(54) DETERMINING DISEASE OR IMMUNE SYSTEM FEATURES BASED ON ANALYSIS OF IMMUNE SYSTEM CHARACTERISTICS

(75) Inventor: Benjamin J. Zeskind, Boston, MA (US)

Correspondence Address: WOLF GREENFIELD & SACKS, P.C., 600 ATLANTIC AVENUE, BOSTON, MA, 02210-2206, US

(73) Assignee: Immuneering Corporation, Cambridge, MA (US)

(21) Appl. No.: 12/718,905

(22) Filed: Mar. 5, 2010

Related U.S. Application Data
(63) Continuation of application No. PCT/US2008/010611, filed on Sep. 11, 2008.
(60) Provisional application No. 60/993,270, filed on Sep. 11, 2007.

(51) Int. Cl. A61K 38/19; G01N 33/53; G01N 33/50; A61K 35/74; A61K 38/21; A61P 37/04; A61B 5/01

(52) U.S. Cl. 424/85.5; 435/7.21; 435/7.24; 424/282.1; 424/278.1; 600/549

(57) ABSTRACT

Methods for analyzing one or more elements, markers, ligands or other characteristics of the immune system of a body (e.g., of a human or other animal), and based on the analysis, determine a location of disease, identify immune system failure, and/or determine treatments based on disease location or immune system failure. All or a part of the immune system process may be analyzed to identify specific features of the immune system, e.g., which may indicate that the disease will evade the immune system and produce a negative outcome. Therapy may be employed to correct immune system failure rather than addressing the disease directly.

### RELATED APPLICATIONS

This application is a continuation of PCT/US2008/010611 filed on Sep. 11, 2008, and claims the benefit of U.S. provisional application Ser. No. 60/993,270, filed on Sep. 11, 2007, the entire disclosures of both of which are incorporated herein by reference.

### BACKGROUND

1. Field of Invention

This invention relates to methods and apparatus for diagnosing and treating human disease, e.g., including infection, allergy, and injury.

2. Related Art

Currently, most human diseases are diagnosed by matching the patient's symptoms to a set of symptoms known to characterize a particular disease. When ambiguity remains, diagnostic tests are used to clarify the diagnosis. For certain diagnoses, patients are treated with oral or injected medicines that spread throughout the body. For other diagnoses, surgery is used to repair or remove damaged tissue.

### SUMMARY OF INVENTION

In accordance with aspects of the invention, information may be extracted from one or more elements, markers, ligands or other characteristics of the immune system of a body (e.g., of a human or other animal) to accomplish one or more of the following goals: determine a location of disease, identify immune system failure, and/or determine treatments based on disease location or immune system failure.

In one aspect, the invention provides a method for diagnosing, and treating human disease by acquiring or otherwise analyzing information from the immune system. In one embodiment, rather than determining a diagnosis based on symptoms characteristic of a disease, diagnosis may instead be determined based on analysis of one or more elements, markers, ligands, or other characteristics of the immune system. For example, all or a part of the immune system process may be assayed, e.g., step by step, to identify specific features of the immune system, e.g., which may indicate that the disease will evade the immune system and produce a negative outcome. In some cases, therapy may begin with an attempt to correct the immune system failure rather than addressing the disease directly, thereby allowing the immune system to neutralize the disease alone. When it is necessary to treat the disease directly, disease location information can be used to select an appropriate therapy and/or to deliver therapy to diseased cells using the location information. As used herein, disease is defined in the broadest sense and can include infection, injury, hyperplasia and other abnormalities, allergy, autoimmunity, cancer, and/or any other condition that may cause harm to a patient.

One aspect of the invention provides a method for determining a location of diseased cells based on information regarding one or more elements, markers, ligands, or other characteristics of the immune system. Disease, injury, and other harmful abnormalities in the body may activate a number of processes through which immune system cells and other components communicate location information to each other and travel to and from the disease site. For instance, cells of the innate immune system send chemical signals that recruit other cells to the site, and also create localized physical differences, such as changes in temperature. Antigen-presenting cells leave the disease site, travel to lymph nodes, and use soluble factors to communicate location information to T-cells. T-cells then express surface receptors and homing behavior that allow them to home to the disease site, aided by chemokine gradients set up at the site. Such information may be used to determine a location of disease, in some cases without requiring a direct observation or other analysis at the specific disease site. For example, in one illustrative embodiment, one or more elements of the immune response (e.g., including cells, soluble factors, chemokine/cytokine concentrations and gradients, ligands of immune system components, physical properties of the body, and so forth) may be analyzed (e.g., by assaying extracted cells for surface molecules or soluble factors secreted, detecting chemokine gradients in vivo, tracking cell movements, determining the presence or absence of immune system cells in a particular location, and so forth). Based on the analysis of the immune system characteristic(s), location information for the disease may be determined, e.g., a detected chemokine gradient may “point” to a disease site. Determining the location of the disease can involve identifying any location-relevant information, including tissue type, actual spatial location, location along a blood vessel, the location of the nearest lymph node, the direction from a given point, the location relative to other cells or tissues, and so forth.

Another aspect of the invention provides a method that identifies immune system failure by analyzing the immune system characteristics of one or more stages of the immune response. In theory, the immune system should neutralize every disease, so when a patient has a negative outcome it is generally because the immune system failed to effectively neutralize the disease. The immune response may include distinct stages beginning with an acute phase response, followed by the action of innate immune system cells such as macrophages and natural killer cells, followed by the action of antigen-presenting cells such as dendritic cells which leave the disease site and migrate to lymph nodes, and activate antigen-specific T-cells which then migrate back to the disease site. One or more stages of this response may be analyzed to identify one or more points of failure that allow (or may allow) the disease to evade neutralization by the immune system or which is causing the immune system to function improperly (as in autoimmune disease).

In another aspect of the invention, knowledge of the immune system failure may be used to design and implement a therapy or regimen of therapies that most effectively corrects the specific immune system failure. Alternately, or in addition, information about the location and/or type of a patient's disease may be utilized for therapeutic purposes. This could include targeting/delivering therapies to diseased cells, modulating the immune system response at certain locations (either increasing or decreasing it), and/or changing the concentration (or adding/removing) immune system elements such as cells, antibodies, and/or cytokines/chemokines.

In one aspect of the invention, a method for determining disease location in a body includes analyzing one or more elements, markers, ligands, or other characteristics of an immune system of a body, and determining a location in the body at which a disease is present based on a result of analyzing the one or more characteristics of the immune system. As discussed above, analysis of immune system characteristics may involve the measurement, assay or other assessment of immune system characteristics, such as cells, cytokines, antibodies, soluble factors, or gradients related to the immune system, as well as the review of previously performed tests (e.g., as may be performed by a clinician after receiving test results from a laboratory). Using information from the analysis, a location for a disease may be determined. For example, immune system cells that display a particular antigen may indicate disease present in the gut, or other specific tissue in the body. As another example, a chemokine or other gradient detected in the body may identify the location of a tumor (e.g., a decreasing chemokine gradient may be associated with the presence of a tumor). As will be better understood from the discussion below, other information regarding immune system characteristics can allow for the determination of disease location.

In one illustrative embodiment, analysis of immune system characteristics may involve analyzing cells by assaying a surface of the cells, extracting cell contents, imaging the cells, assaying factors secreted by the cells (such as cytokines that recruit other cells or factors that imprint tissue-specific homing properties onto other cells of the immune system), tracking a migratory behavior of the cells, or assaying one or more binding characteristics of the cells. Assaying a surface of the cells may be done by assaying one or more of T-cell receptors, MHC-antigen complexes, integrins, selectin ligands, chemokine receptors, B-cell receptors, KIR receptors, cytokine receptors, or NKG2 receptors, for example.

Another aspect of the invention relates to identifying failure of the immune system in responding to cancer. For example, a method for detecting failure of one or more phases of an immune system includes analyzing one or more characteristics of the immune system of a body during a response of the immune system to a cancer in the body, and determining failure of at least one phase of the immune system in responding to the cancer based on a result of analyzing the one or more characteristics of the immune system. Analysis of the immune system characteristics may involve assessing a function of the immune system at one or more phases of its sequential process. For example, one or more characteristics of the immune system associated with activation of the innate immune system, activation of the adaptive immune system, acute phase response, behavior of macrophages, natural killer cell response, regulatory T cell behavior, interaction of cells via integrins, recruitment and activation of antigen presenting cells, migration of antigen presenting cells, activation of T cells, tissue specific homing of T-cells, and/or disease neutralization activity of T cells may be analyzed, and a function of the immune system determined based on the analysis. Characteristics of the immune system that may be analyzed include, for example, determining a number of natural killer cells present, determining characteristics (such as phenotype, expression level, concentration, and/or binding affinity) of natural killer cell activating, inhibitory, and/or binding receptors and/or the ligands of the receptors, and so on. In one embodiment, a disease outcome with respect to the cancer may be predicted based on a result of determining a failure of a phase of the immune system.

In another aspect of the invention, a method for developing and/or implementing a treatment for disease includes analyzing one or more elements, markers, ligands, or other characteristics of an immune system of a body, determining a location of the body at which a disease is present and/or immune system failure based on a result of analyzing the one or more characteristics of the immune system, and determining a treatment for a disease based on information about disease location and/or immune system failure. As mentioned above, disease location may be used to target or deliver a therapy to specific cells or tissues. Similarly, information about immune system failure may be used to devise a treatment that specifically corrects the immune system failure, and enables the immune system to neutralize the disease. For example, a therapy that specifically corrects the immune system failure may include an addition or removal of cells, cytokines, hormones, antibodies, proteins, or other molecules with respect to the body, blocking of activating or inhibitory receptors on immune cells in the body, and/or promoting a likelihood of a cytotoxic reaction to cancer cells.

These and other aspects of the invention will be apparent from the following detailed description. It should also be understood that the many aspects of the invention may be used alone and/or in any suitable combination with other aspects of the invention.

### BRIEF DESCRIPTION OF THE DRAWINGS

Aspects of the invention are described with reference to illustrative embodiments and the following drawings, in which like numerals reference like elements, and wherein:

FIG. 1 shows steps in a method that incorporates aspects of the invention in an illustrative embodiment;

FIG. 2 shows a schematic of a first assay for use in determining disease type and location in an illustrative embodiment;

FIG. 3 shows a schematic of a second assay for use in determining disease type and location in an illustrative embodiment; and

FIG. 4 shows steps in a method for analyzing immune system characteristics and determining an immune state in an illustrative embodiment.

### DETAILED DESCRIPTION

Various aspects of the invention are described below in relation to specific illustrative embodiments, but it should be understood that the various systems and methods described herein are applicable to other applications, such as use with animals other than humans. Also, aspects of the invention may be used alone and/or in any suitable combination with each other. For example, aspects of the invention related to determining disease location based on analysis of immune system characteristics may be combined with aspects of the invention related to determining failure of one or more phases of immune response, and/or with aspects of the invention related to developing a therapy based on disease type or location information, etc.

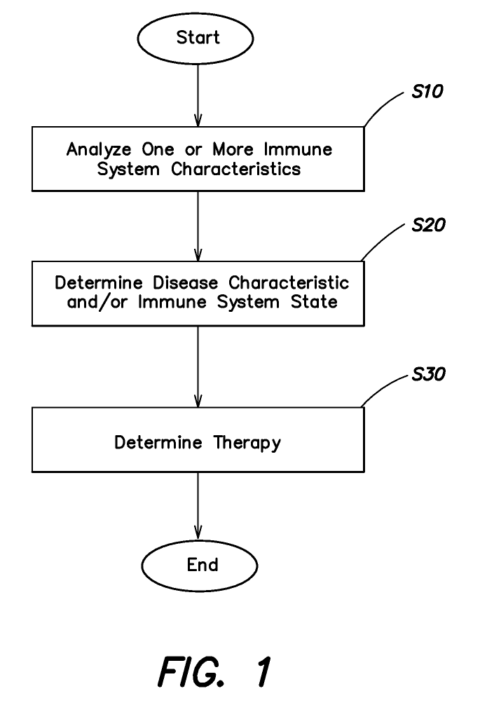

FIG. 1 depicts steps that may be performed in one illustrative method that incorporates aspects of the invention. In step S10, one or more elements, markers, ligands, or other characteristics of the immune system may be analyzed. For example, one or more characteristics of the immune system may be monitored to detect changes in the characteristics, which may indicate the presence of disease in the body. (In some cases, changes in immune system characteristics may indicate the presence of disease prior to the onset of symptoms consistent with the disease.) This could involve analysis of one or more characteristics related to one or more of the following: activation of the acute phase response of the immune system, activation of the innate immune system, activation of the complement system, inflammation, or other signs of immune system activation. Such analysis could involve, for example, measuring the concentration of protein or other substances in the blood, lymph, urine, saliva, breath, skin headspace gas, or other samples, and/or noninvasively analyzing protein concentrations using spectroscopy or other means, and/or noninvasive detection of other characteristics or physical signs of immune system activation including, but not limited to, changes in physiological temperature. Moreover, analysis performed in step S10 may be done by a review of test results, e.g., as may be done by a clinician reviewing test results from a laboratory. For example, a doctor may receive results from laboratory that indicates protein concentrations or other measurement data of compounds associated with immune system function, and analyze the test results. In another illustrative embodiment, a suitably programmed general purpose computer (e.g., operating one or more software modules that constitute an expert system or other arrangement suitable for analyzing data) may receive test results of other data and analyze the data accordingly.

Based on the analysis of immune system characteristics in step S10, a disease characteristic (such as the disease type and/or location) or an immune system state (such as a failure in one or more phases of immune system response) may be determined in step S20. As discussed in detail below, immune system characteristics can be used to determine the type of disease (e.g., an antigen against which the immune system is activated may identify a type or set of types of diseases), a location for disease (e.g., chemokine gradients in the body may indicate a location where diseased cells are generally clustered), and/or failure of the immune system (e.g., the presence of disease in the absence of a C-reactive protein response may indicate a failure of the acute phase immune system response). Using the examples above, a clinician may analyze test result data regarding immune system characteristics and determine a location for disease, a failure of one or more phases of the immune response, etc. Similarly, an expert system or other computer program operating on a data processor may use test result data or other information to determine disease location, immune system failure, etc. Criteria that may be used to make such determinations are discussed in more detail below.

Next, in step S30, information on disease type or location, and/or immune system failure may be used to determine a treatment for the disease or other therapy. For example, a tumor may act as a chemokine sink, thereby effectively avoiding targeting by the immune system. If a determination is made that a chemokine gradient suitable to aid in immune system targeting of tumor cells is absent, a therapy may be developed by which a suitable chemokine gradient is artificially introduced at the tumor site to enhance T-cell and other immune system activity.

Below, details regarding various options for performing steps of the method shown in FIG. 1 are described. However, it should be understood that the various steps performed in the method of FIG. 1 may be eliminated in some aspects of the invention and/or various steps may be used alone and/or in any suitable combination with steps shown or not shown in FIG. 1. Thus, the method outlined in FIG. 1 should not be interpreted as limiting aspects of the invention in any way.

Analysis of Immune System Characteristics

As suggested above, the analysis of immune system characteristics in step S10 of the method of FIG. 1 may involve the identification, measurement or other analysis of various compounds, cells or other characteristics associated with the immune system and its function. For example, in one illustrative embodiment, characteristics of the immune system related to the system's acute phase response may be analyzed. The human immune system responds rapidly to disease by a mechanism called the acute phase response, which includes a nonspecific physiological and biochemical response to tissue damage, infection, inflammation, and/or malignant neoplasia. For instance, the concentration of C-reactive protein in the blood can increase 10,000× in a matter of hours during the acute phase response. Thus, analysis of C-reactive protein in a body may reveal protein levels that may indicate activation of the acute phase response (and which may be used in determining a disease characteristic or immune system state in step S20). Although C-reactive protein is discussed below, any other protein or other material indicative of acute phase response, including serum amyloid A (SAA), could be analyzed instead or in addition in this illustrative embodiment. Of course, other immune system characteristics could be analyzed when assessing other immune system response phases or disease features.

Analysis of immune system characteristics could be performed in any suitable way. For example, analysis of an immune system characteristic may indicate the presence or absence of a compound (e.g., the presence of a C-reactive protein), a concentration of a compound (e.g., a concentration of C-reactive protein in a particular body area), a gradient of a compound (e.g., a concentration gradient of C-reactive protein within a particular body area), the presence or absence of a particular immune system cell or feature of the cell (e.g., a presence of cells displaying a particular antigen), and so on. The immune system characteristics may be analyzed directly at the body and/or using samples taken from the body. For example, patients could self-collect a small liquid sample of blood or urine weekly, and the samples could be collected and be transported to a laboratory to determine the concentration of C-reactive protein in the sample using standard commercially available test kits. Alternately, the patient could provide the blood or urine sample directly to a home-based electronic device that could determine the concentration of C-reactive protein in the sample. In either case, records regarding concentration levels could be kept electronically, and the patient or the patient's physician could be notified of the results. A physician could perform an analysis of the of the recorded concentration levels, e.g., by reviewing the results displayed on a personal digital assistant (PDA) or other electronic device.

In another embodiment, patients could self-collect a small gaseous sample of breath by blowing into a tube with a one-way valve. The sample could be analyzed for signs of immune system activation and/or disease characteristics. For example, a sign of immune activation could include a suitable ethane level in the breath, since ethane levels in exhaled breath have been shown to correlate with a patient's C-reactive protein levels. Alternately, the patient could provide the breath sample directly to a home-based electronic device that would determine the concentration of ethane, and store and/or send results of the analysis. In another embodiment, a non-invasive detector of acute phase response could be worn constantly, like a watch with built in heart rate monitor, or a patient could be scanned daily at home with a non-invasive device. In another specific embodiment, the serum concentrations of soluble ligands of immune system receptors could be monitored. For instance, the concentrations of MICA or ULBP2 (both ligands of NKG2D) could be analyzed using a suitable device and/or technique.

Once one or more immune system characteristics have been analyzed, a disease characteristic, such as the disease type or location, and/or an immune system state (e.g., a level of failure of one or more phases of the immune response) may be determined based on results of analyzing the one or more characteristics of the immune system. Currently diseases are diagnosed by matching the patient's symptoms to a set of symptoms known to characterize a particular condition. When ambiguity remains, diagnostic tests are used to clarify the diagnosis and imaging techniques may be used to locate the disease. This approach is inherently qualitative, and focused on the end result of the disease rather than its cause. Yet virtually every disease involves a specific abnormality in a specific group of cells. Techniques in accordance with the invention could be used whether a patient requires diagnosis because of the appearance of symptoms, or because of the detection of immune system activation as described above.

Determining Disease Type

As mentioned above, determining a disease type can involve the identification of injury, inflammation, allergy, hyperplasia, cancer or other disease (as well as a type under any one of these categories, such as a specific type of cancer). In one embodiment, determining the disease type can be viewed as a specific cellular abnormality causing the disease. For example, determining the disease type may be done based on the results of analysis of the immune system characteristics, e.g., based on identifying the antigen against which the immune system is activated, identifying cancerous cells, identifying inappropriate cytokine levels, or identifying any other factor or characteristic that helps determine the disease type.

In one specific embodiment, a disease type may be determined by detecting the antigen against which antigen-presenting cells, such as dendritic cells, are activated. In most diseases, when the immune system is activated against the disease, antigen-presenting cells including but not limited to dendritic cells and macrophages will endocytose antigens and then display the antigens in class II MHC molecules on the surface of the antigen-presenting cell. Activated dendritic cells leave the abnormal cells, enter the lymph (or blood), and home to lymph nodes where they can then present antigen to helper T cells and cytotoxic T lymphoctyes. These activated dendritic cells contain information that could be assayed to determine the type of disease. For instance, these antigen-presenting cells may be sampled to determine which antigen they are presenting, using currently existing technology to recognize specific MHC-peptide complexes using recombinant T-cell receptors in soluble form. For example, an array could be created from recombinant T-cell receptors for antigens associated with known diseases. A blood, lymph, or other sample may be taken and dendritic cells separated from other cell types, e.g., using fluorescence-activated cell sorting or other means. The activated dendritic cells may then be exposed to the array, and the number of dendritic cells binding at any given location will indicate the extent to which the immune system is activated against the particular antigen at that address on the array. The antigens making up the array could include any known to be specific for human disease, including but not limited to: tumor specific antigens (for cancer), self antigens (for autoimmune disease), oxidized LDL (for atherosclerosis), amyloid (for Alzheimer's), myelin (for multiple sclerosis), cartilage (for rheumatoid arthritis), and any other disease-related antigen.

In another specific embodiment, the disease type may be determined based on detecting the antigen against which T-cells are activated. Once activated, dendritic cells travel through the body to the lymph nodes, they activate T-cells with receptors specific to the disease antigen, and these T-cells multiply due to clonal selection before leaving the lymph node to travel to the site of disease. In one embodiment, a disease type could be determined based on an analysis of activated T-cells. The T-cells could be analyzed to determine the antigen against which they are activated using technology that currently exists to recognize specific T-cell receptors using a live-cell MHC-peptide array to identify those antigens against which a large number of T-cells are activated. The T-cells could be sampled from blood, lymph, or other sample taken from the body and then sorted using commercially-available fluorescence-activated cell sorting (FACS) system to separate CD4⁺ (Th) cells and CD8⁺ (CTL) cells. A live-cell MHC-peptide array may be used to determine antigen(s) against which large numbers of T-cells are activated. T-cells with receptors specific to the disease-causing antigen will typically be present in larger numbers due to clonal selection. This could be done instead of, or in addition to, the antigen-presenting cell assay described above.

In another illustrative embodiment, the disease type may be determined by assaying B-cells or the antibodies they produce. When B-cells become activated, they may become plasma cells and travel to the spleen or bone marrow, making the B-cells difficult to sample. However, other B-cells may remain in the blood while traveling to other lymph nodes. Isolating B-cells from the blood, lymph, or other sample and assaying their B-cell receptors may make it possible to determine the antigen against which they are activated and thus identify the disease type. Alternately, the antibodies that B-cells produce could be sampled from the blood as a means to determine the antigen against which the immune system is activated and thereby identify the disease type.

In another specific embodiment, the disease type may be determined by detecting IgE antibodies or mast cells with IgE molecules on their surface with specificity against common allergens.

In another embodiment, the disease type may be determined based on an analysis of macrophages. Like dendritic cells, macrophages are antigen presenting cells and thus could be used to determine the antigen against which the immune system is activated. While dendritic cells travel through the bloodstream from the diseased site to lymph nodes, macrophages remain at the disease site and thus may be more difficult to sample, especially when the disease site is unknown. However, if the disease site is known (as discussed in more detail below) or areas of inflammation can be detected and sampled, macrophages can provide valuable information. For instance, macrophages could be sampled from sites within tissue. These sites could be determined because the disease location is known, or sites of inflammation could be detected and sampled, or areas of tissues sampled randomly, or a location chosen by any other means. Macrophages could be separated from other cells and exposed to an array created from recombinant T-cell receptors for antigens associated with known diseases. The number of macrophages binding at any given location may indicate the extent to which the immune system is activated against the particular antigen that is present at that address on the array. The antigens making up the array could include any known to be specific for human disease, including but not limited to: tumor specific antigens including phosphatidylserine (for cancer), self antigens (for autoimmune disease), oxidized LDL (for atherosclerosis), amyloid (for Alzheimer's), myelin (for multiple sclerosis), cartilage (for rheumatoid arthritis), and any other disease-related antigen.

In another embodiment, the disease type may be determined by measuring other characteristics of the immune system such as cytokine and/or chemokine levels. For example, sepsis can be detected by measuring levels of tumor necrosis factor (TNF), IL-12, or IFN-gamma in the body. Chemokines and cytokines are used by the body to control immune responses, so knowledge of their presence, concentration, and dynamics can be used to identify the immune system's state of activation (as discussed in more detail below). This could indicate that the immune system is not sufficiently activated, or that it is excessively activated as in the case of autoimmune disease.

In another embodiment, disease type may be determined by examining T-cells and B-cells for abnormalities. For instance, lymphoma can be identified by detecting changes in B-cells and T-cells indicative of cancer.

In another embodiment, disease type may be determined by examining ligands of immune system elements. For instance, elevated levels of soluble MICA (a ligand of NKG2D) have been detected in serum from multiple myeloma patients.

These are just a few illustrative examples of one aspect of the invention which involves determining disease type based on an analysis of one or more elements, markers, ligands, or other characteristics of the immune system. It should also be understood that the many aspects of the invention may be used alone and/or in any suitable combination with other aspects of the invention. For instance, antigen presenting cells could be analyzed for the antigen they are presenting, AND T-cells could be analyzed for the antigen against which they are activated. This approach could lend additional accuracy to the diagnosis of the abnormality, and could also be used to identify cases of immune system failure in which antigen presenting cells effectively detect the disease, but T-cells are not properly activated against these antigens.

Determining Disease Location

In another aspect of the invention, a method for diagnosing a patient's condition involves determining the disease location based on an analysis of one or more elements, markers, ligands, or other characteristics of the immune system. To fully diagnose a disease it may be important to know not only what abnormality is occurring (as represented by the disease type), but also in which cells this abnormality is occurring (as represented by the disease location). Any information that can be gleaned about the type of cell that is diseased or its location in the body may be useful both for diagnosis and treatment. Location can be defined as a tissue type, a nearest lymph node, a place along a blood vessel, a location within a specific coordinate system (whether in 3-dimensions or fewer), or any other information that helps locate the disease spatially or in relation to other cells, tissues, or organs.

In one illustrative embodiment, location of the disease may be determined based on an analysis of antigen-presenting cells. When antigen presenting cells such as dendritic cells travel to a lymph node in order to activate T-cells, they must communicate sufficient information to the T-cells to enable them to traffic back to the disease site. Soluble factors produced by dendritic cells from different sources as well as other factors in tissue microenvironments can provide tissue-specific homing properties to responding T cells. For example, dendritic cells from the intestinal lymphoid tissue produce retinoic acid, which is not made by dendritic cells from other sources, and has been found to influence T-cells in their movement to a disease site. By sampling antigen-presenting cells from the blood, lymph, or other sample and assaying them for factors (both soluble and cell-surface) that convey tissue specific homing properties on T-cells, the location of the disease can be determined. This can include factors currently known as well as those yet to be discovered. This assay can be done either in conjunction with determining the antigen against which they are activated (described above) or separately. A blood, lymph, or other sample can be taken and dendritic cells separated from other sample components using fluorescence-activated cell sorting or other means. The dendritic cells can then be assayed to determine if are producing retinoic acid, in order to determine whether or not the diseased cells are in the gut, for example.

In another embodiment, additional factors through which dendritic cells convey tissue specific homing properties onto T-cells can be determined by assaying a dendritic cell for a variety of soluble factors, then exposing the dendritic cell to a T-cell, and tracking the tissue homing behavior of the T-cell using a means such as fluorescence or MRI of SPIO (superparamagnetic iron oxide particles) endocytosed by T-cells (a similar procedure has been used to track dendritic cells with resolution around 500 μm in vivo).

In another embodiment, location of the disease is determined based on assaying T cells. As mentioned above, T cells display tissue-specific homing properties. The T-cells activated against a disease-causing antigen will also contain chemokine receptors, integrins, and selectin ligands specific to the location of disease. For example, it has been found that the recruitment of leukocytes to a site of infection or tissue injury involves a variety of cellular adhesive events, including tethering, rolling, adhesion, diapedesis, transmigration and chemotaxis. Selectins help to control leukocyte tethering and rolling, thereby bringing circulating leukocytes into proximity with extracellular stimuli such as cytokines, chemokines and chemoattractants that are displayed on or released from activated endothelium. These extracellular stimuli activate integrins, which help leukocytes adhere at the disease site. T cells may be assayed in order to identify the location of diseased tissue based on tissue-specific homing properties of the cell. For example, a blood, lymph, or other sample may be obtained and the T cells extracted using FACS. The T cells could then be assayed and those that localize to inflamed skin may be identified by the presence of E-selectin ligands, P-selectin ligands, chemokine receptors CCR4 and CCR10, and/or CCR-17 and CCR-22. T cells that localize to small intestinal lamina propria and mucosal epithelium may be identified by the presence of α4β7 integrin, CCR9, and CCR25. T cells that localize to epidermis may be identified by the presence of integrin α₁β₁ (very late antigen VLA-1). In this fashion, a live cell array could be built that would identify the specific tissue locations against which the T-cells are activated.

In another embodiment, location of the disease is determined by identifying additional markers of T-cell tissue specific homing using the following method: assaying the T-cells for various surface markers and then reinjecting them into the body and tracking their migration using a means such as fluorescence or MRI of SPIO (superparamagnetic iron oxide particles) endocytosed by T-cells (a similar procedure has been used to track dendritic cells with resolution around 500 μm in vivo).

In another embodiment, disease location is determined based on analyzing gradients. Once T cells tether and roll along the inside of a blood vessel, they detect gradients of cytokines and other soluble factors in order to express integrins, adhere to the vessel wall, extravasate into tissue, and home to the site of abnormality. To fully determine the identity of diseased cells it may be necessary to detect and map these gradients within the body. These context cues not only stimulate cells to move, but can also influence certain cells to differentiate, proliferate, or to undergo apoptosis. Just as important as the presence of soluble factors may be their absence. Tumors create a tryptophan “sink” around themselves; an area of low tryptophan concentration that hinders T-cell proliferation. Other soluble gradients can play an important role in wound repair by providing cells with the context cues necessary to differentiate. For instance, in neurons, gradients of the same compound can act as both a morphogen, specifying cell differentiation, and a guidance cue, directing axon movement. For example, one compound, Shh, can function as both a morphogen and a chemoattractant.

Concentration gradients of soluble factors within the body can be detected and quantified as part of immune system diagnosis. Some embodiments use chemokine gradients to identify and localize disease, and could detect these gradients using any current technology or a technology yet to be invented. One soluble factor of interest is the IL-2 that is produced by activated helper T cells. For instance, fluorescent antibodies to IL-2 or other cytokines could be used to identify concentration gradients and are imaged using a fluorescent endoscope or “Pill cam” style device. Alternately, near infrared (NIR) fluorescent polymersomes, which allow imaging of tissues at centimeter tissue depths could be coated with antibodies to the appropriate cytokine and used to image in vivo using an endoscope or other instrument. Alternately, native fluorescence imaging of tryptophan could be used in conjunction with an endoscope or “Pill-cam” style device to detect gradients of tryptophan and other factors in vivo. Alternately, a device could be used to detect gradients using motion, as bacteria do. Alternately, extremely small radio-frequency identification (RFID) microchips, which transmit a unique identifier when activated by an RF signal, could be coated with antibodies to the appropriate cytokine and used to detect the presence or concentration of cytokines in vivo and the precise position located through signal triangulation. Alternately, a flat adhesive surface could be placed against an area of tissue and then removed, and the concentration of the desired molecule examined at various spatial locations on the adhesive surface. Alternately, a long adhesive needle could be inserted (perhaps radially to a blood vessel), and then removed and the concentration of the desired molecule examined at various spatial locations along the adhesive needle.

In another embodiment, location of the disease could be determined based on analyzing areas of inflammation within the body. For instance, areas of inflammation could be detected by identifying areas of higher temperature within the body within a localized area. For example, temperature variations could be detected with a spatial resolution of about 500 micrometers or greater. (By “resolution about 500 micrometers or greater,” it is meant that a resolution of the temperature detection process is capable of determining a temperature of a region along a 1-dimensional line, within a 2-dimensional area or 3-dimensional volume, having a largest dimension of 500 μm or less. For example, if the temperature detection is performed in 2-dimensions, a temperature may be determined for a plurality of square or otherwise shaped “pixels” having a largest dimension of about 500 micrometers or less.) The variations in temperature could allow for the identification of a disease location to a resolution of 500 micrometers or greater. This could be done using high resolution infrared imaging, direct contact thermal probes, or any other suitable means. The inflamed plaques associated with atherosclerosis are believed to have higher temperature and arterial wall thermography may be used to map temperature differences in order to locate these plaques.

In another embodiment, location of the disease could be determined by sampling tissue at a given location and analyzing that tissue for macrophages activated against the diseased antigen. The presence of these macrophages could confirm the location. Alternately, tissue in a number of locations could be sampled for macrophages activated against the disease. If macrophages activated against the disease-causing antigen are detected in one of the samples, the origin of that sample provides information about the location of the disease.

In another embodiment, location of the disease could be determined by taking blood, lymph, or other samples for the assays described above from various locations in the body, and the sample with the highest concentration of activated cells may be determined likely to be closest to the site of disease, for example.

In another embodiment, location of the disease could be determined by identifying the specific lymph node most activated and thus closest to the disease site.

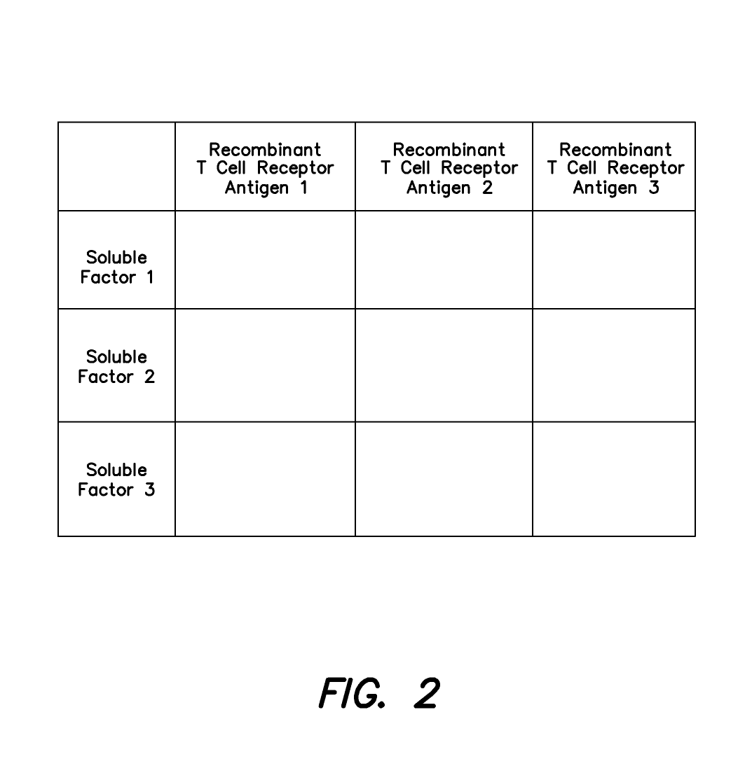

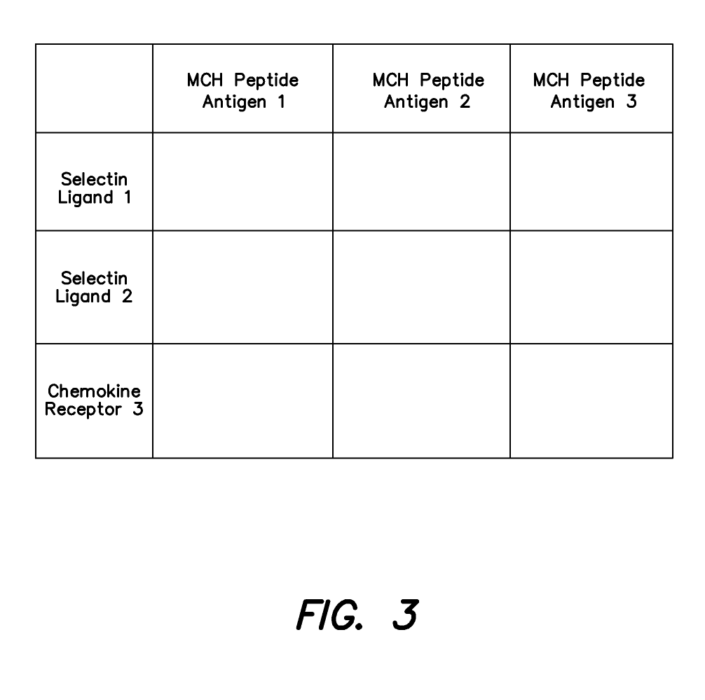

In another aspect of the invention, the type of disease and location of the disease could be identified simultaneously. In one specific embodiment, dendritic cells are analyzed using an array such as that depicted in FIG. 2, in which recombinant T-cell receptors are used for assaying disease type by means of antigen specificity, while soluble factors are assayed to determine disease location. A two dimensional grid could be used in which the rows represent specific antigens and the columns represent specific locations. In another embodiment, T-cells may be analyzed by combining a location array with the MHC-peptide array described above; for instance, the rows of the array could correspond to antigen and the columns to the address. Such an array is depicted in FIG. 3.

Determining Immune System Failure

In another aspect of the invention, one or more failures of the immune system may be identified. In one embodiment, failure of the immune system in one or more phases may be determined in the case where a body has one or more cancerous cells or tissues. Such failures of the immune system enable diseases to evade the immune system and escape neutralization. Traditionally, once a disease is diagnosed it remains difficult to predict the disease outcome. Often little more is known than the percentages of patients with favorable and unfavorable outcomes. This uncertainty about the disease outcome may make it difficult to decide whether or not to initiate a treatment, especially since treatments often bring additional risks and damaging side effects. In theory, every patient's immune system should be able to control any disease and produce a favorable outcome. So while a negative outcome for a patient has traditionally been viewed as a result of an offensive disease, in this aspect of the invention a negative outcome is viewed more as a failure of the patient's own immune system.

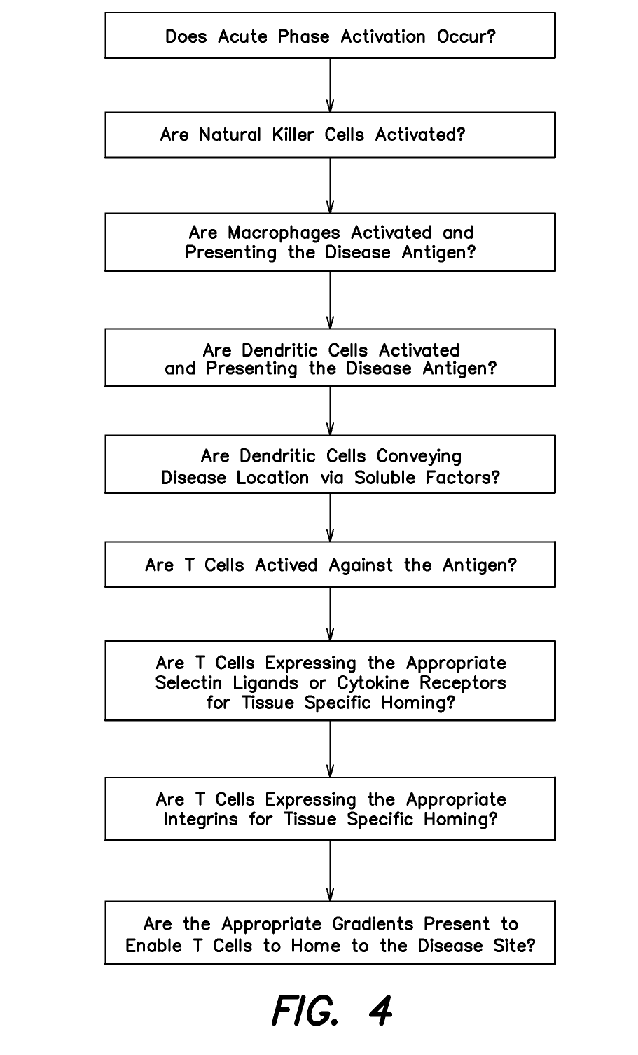

In one embodiment, immune system failures may be identified by assessing the function of the immune system at each phase, or at one or more phases, of its sequential process. Proper immune functioning, from acute phase response to CTL tissue-specific homing, may be used to predict a favorable outcome and suggest that there is no need to treat. A failure of the immune system at any specific point in the process may be used to predict an unfavorable outcome and may increase the necessity to treat. A system to identify immune system failures could provide this medically relevant information. For instance, if analysis of the acute phase response (e.g., using C-reactive protein levels or other means) does not detect a response, but a disease occurs anyway, then the innate immune system may be determined as failing to detect the disease. If the acute phase response occurs but no natural killer cells are activated against the disease, then there may be a failure in natural killer cell activation. If no dendritic cells are found to be activated against the disease-causing antigen (using recombinant T-cell receptor arrays or other means), then there may be a failure in the process of dendritic cell activation or antigen presentation. This can be further investigated by lysing dendritic cells. If they are found to contain the disease-causing antigen, but are not presenting it on their surface, the problem may be specific to antigen presentation. (If macrophage samples are available, this can be used to determine whether or not macrophages are activated against the antigen, presenting the antigen, and producing the cytokines necessary to attract and activate dendritic cells. Macrophages could also be lysed to determine if they have phagocytosed an antigen, but are not presenting it). If dendritic cells are found to be activated against the antigen, but not to convey soluble signals of tissue specific homing, then there may be a failure in the process by which dendritic cells convey location information to T-cells. If dendritic cells are activated against the disease specific antigen and convey soluble signals of tissue specific homing, but T-cells are not detected activated against the antigen, then there may be a failure in process by which dendritic cells find and convey information to the appropriate T-cell. If T-cells are activated against the antigen but not expressing the appropriate selectin ligands or cytokine receptors, there may be a failure in the process by which these receptors are upregulated. If T-cells are activated against the antigen and expressing the appropriate selectin ligands and cytokine receptors, but are not expressing the proper integrins, there may be a failure in the process by which these molecules are upregulated. If the appropriate chemokine gradients are not present in the tissue to allow the T-cells to home to the site of injury, and they can be added, there may be a failure in this process. FIG. 4 illustrates an example of this approach to identifying immune system failures that may be employed as part of step S10 in the FIG. 1 process of analyzing immune system characteristics. Based on the results of analysis at one or more steps, a determination may be made regarding the failure of the immune system response. In addition to the steps described above, this method also includes any other method of analyzing the immune system at multiple points in order to identify the specific point at which an immunological failure is occurring.

In one illustrative embodiment, immune system failure involves the inability of natural killer cells to develop a cytotoxic reaction to diseased cells (e.g., cancerous cells), or the development of an inappropriate cytotoxic reaction against healthy cells. Natural killer cell behavior is driven by the signals received from activating and inhibitory receptors. These receptors bind to ligands that may be present on the surface of a target cell (whether diseased or healthy) such as the human leukocyte antigen (HLA) molecules, and also bind to ligands that are present in soluble form in the microenvironment. Such soluble ligands include cytokines such as interleukins, interferons, as well as tumor growth factors (TGF), tumor necrosis factors (TNF), and other soluble ligands such as soluble human leukocyte antigen g (sHLA-G) and soluble major histocompatibility complex class I-related chain A (sMICA). Natural killer cell activating and inhibitory receptors also bind to the Fc ends of antibodies.

In one embodiment, the activating and inhibitory receptors and their ligands (as well as the number of NK cells) may be analyzed for properties including expression level, concentration, phenotype, binding affinity, and trafficking characteristics such as internalization and degradation rates. For instance, a tumor may downregulate the expression of those activating ligands for which a patient's natural killer cells have activating receptors. Identifying immune system failures may include identifying the inability of the immune system to overcome particular tumor immune evasion mechanisms such as the one described above.

Immune system failures may also include the inability of the immune system to become activated against the Fc end of a therapeutic antibody. This is also referred to as an antibody-dependent cellular cytotoxicity (ADCC) response.

In another embodiment, immune system failure may include failures involving integrins and their ligands and other molecules that enable natural killer cells to spatially localize to a target cell, remain in contact with it, and form an immunological synapse.

In another embodiment, the immune system failure may be used to classify, categorize, or describe the disease that is caused by the immune system failure or that causes the immune system failure. For instance, an individual patient's cancer is traditionally classified by the tissue of origin (breast, prostate, etc.) but could instead be classified by the cellular and molecular methods that it uses to evade the immune system. Such a classification could be dynamic, changing over time as an individual's cancer develops new immune evasion mechanisms. This new means of classifying cancer would facilitate the selection of therapeutic regimens that can activate an individual patient's immune system against their particular tumor.

Determining Therapy

In another aspect of the invention, information about disease type, disease location, and/or immune system failure may be utilized to develop specific and targeted treatments, e.g., as part of step S30 in the method of FIG. 1.

In one specific embodiment, immune system failures that have been identified may be systematically corrected in order to enable the immune system to successfully neutralize disease, if present. For instance, if a disease fails to induce an acute phase response, methods can be found to activate the complement system against the specific antigen. This could include isolating a disease-causing cell and coating it with lipopolysaccharide, mannose, or another molecule that is known to activate the complement system. Alternately, if the acute phase response occurs but no dendritic cells are found to be activated against the disease-causing antigen, then therapeutic methods could be used to artificially activate dendritic cells against the antigen. This could include isolating dendritic cells from the blood, lymph, or other sample, exposing them to the antigen in the presence of activating cytokines, then reinjecting them into the body. Alternately, if macrophage information is available and macrophages are failing to be activated, then a substance could be injected to the disease site to activate the macrophages. This could be a bacterium such as BCG, a cytokine like IFN-gamma, or any other way of activating macrophages. Alternately, if dendritic cells are found to be activated against the antigen, but not to convey soluble signals of tissue specific homing, then such soluble signals could be provided externally while stimulating T-cells with the dendritic cells either externally or in an artificial lymph node. Alternately, if dendritic cells are activated against the disease specific antigen and convey soluble signals of tissue specific homing, but T-cells are not detected as being activated against the antigen, such T-cells can be isolated and activated externally or in an artificial lymph node. Alternately, if T-cells are activated against the antigen but not expressing the appropriate selectin ligands or cytokine receptors, they can be artificially activated to do so. This could include exposing them to cytokines or soluble factors that increase the expression of these surface molecules. Alternately, if T-cells are activated against the antigen and expressing the appropriate selectin ligands and cytokine receptors, but are not expressing the proper integrins, cytokines can be used to artificially upregulate these integrins. Alternately, if the appropriate chemokine gradients are not present in the tissue to allow the T-cells to home to the site of injury, they can be added artificially using a device or a cell.

In another embodiment, if the immune system failure is determined to involve insufficient or excessive activation of natural killer cells, that particular failure is corrected. This may be corrected by providing or removing one or more cytokines or other ligands for activating receptors of natural killer cells, and/or by providing one or more cytokines or other ligands for the inhibitory receptors of natural killer cells and/or by blocking one or more of these receptors. For instance, Interleukin-2 and Interferon Alpha may be administered to activate natural killer cells at the same time that anti-KIR antibody is administered to overcome inhibition.

In another embodiment, an immune system failure may be corrected in whole or in part by lowering the expression levels of inhibitory KIR receptors. This may be achieved by lowering a patient's psychological stress levels or by any other means.

In another embodiment, an immune system failure may be corrected in whole or in part by increasing or decreasing the levels of zinc or any other molecule that modifies the binding affinities of immune system receptors.

In addition to the steps described above, the method may include any other approach to treating an identified immune system failure in order to enable the immune system to successfully fight a disease.

In another embodiment, information about the type of disease may be used to develop a treatment. For instance, if the particular antigen causing the disease has been identified, then antibodies against that antigen could be created and delivered, or the population of immune system cells specific to the desired antigen could be clonally expanded. Alternately, undesired immune system cells or cytokines can be removed and/or eliminated. These cells could include T-cells activated against myelin (and thus causing multiple sclerosis), cartilage (and thus causing rheumatoid arthritis), beta cells (and thus causing type I diabetes), other self-antigens (causing other autoimmune diseases), mast cells with IgE molecules against allergens (to prevent allergy), oxidized LDL (and thus causing atherosclerosis) or any other undesired immune system cell. Alternately, undesired immune system cells or cytokines can be filtered from the blood, lymph, or other sample using a dialysis-like instrument that removes blood, lymph, or other sample from the body, passes it over a filter containing self-antigen molecules such as myelin or cartilage, allergens, or other ways to bind undesired immune system cells, and then passes it back into the body. If the number of helper T-cells and cytotoxic T-lymphocytes can be sufficiently reduced, the positive feedback loop causing the response can be stopped. In order to increase the adhesion of desired cells, the filter could be coated with selectins, intercellular adhesion molecules or other integrin ligands, or chemokines that would induce the T-cells to upregulate their integrins. Using the arrays and other methods described earlier to determine the antigen and address against which T-cells are activated, the specific address against which the autoimmune T-cells are activated could be determined and used to increase the affinity of the filter by choosing the correct selectins, ICAMs, and chemokines. Alternately, a stent-like or other implantable device may be inserted into the bloodstream and used to bind, trap, and kill cells activated against self antigen. Alternately, tumor necrosis factor (TNF), IL-12, or IFN-gamma could be filtered from the blood, lymph, or other sample in order to prevent sepsis and septic shock. This could be done using a dialysis-like instrument that removes blood, lymph, or other sample from the body, passes it over a filter, and then passes it back into the body. By removing enough of these cytokines, the positive feedback loop that causes sepsis could be halted.

In another embodiment, cytokines and/or chemokines could be added, removed, or have their concentrations altered in one or more specific locations in the body. These could be used to activate the immune system, deactivate it, or modulate the intensity of its response.

In another embodiment, ligands of immune system cells could be added, removed, or have their concentrations altered in one or more specific locations in the body. These could be used to activate the immune system, deactivate it, or modulate the intensity of its response.

In another embodiment, knowledge of the location of diseased cells could be used to develop therapies. Most treatments are currently delivered either orally or via injection and expose all the cells of the body, having some beneficial effects on the disease cells but often causing side effects on other cells. The additional address/location information obtained in diagnosis may enable the more precise targeting of therapies specifically to the diseased cells in order to both increase the efficacy of the treatment and also avoid side effects. For instance, gradients of chemokines, morpohogens, or other factors could be created either directly, using an in vivo device, via a controlled-release microsphere, via a cell, or using another method. Alternately, disease location information could be used to create microspheres that contain a therapeutic and are also targeted to bind to the appropriate tissue type. Alternately, therapy could be delivered using leukocyte mimetic microspheres coated with appropriate chemokine receptors, integrins, and selectin ligands for the disease location. Alternately, tissue specific homing could be used in general to deliver therapy to the diseased cells. Alternately, a T cell targeted to the diseased area could be loaded with a therapeutic microparticle and re-injected into the body. Alternately, a T cell can be exposed to cytokines to externally upregulate specific integrins in order to ensure that the t-cell homes to the specific tissue where the disease occurs. Alternately, therapies could be targeted to the lymph nodes as therapies for lymphoma in which the disease resides in these nodes. Alternately, chemokine or morphogen gradients could be created within the body to aid immune cells in locating the diseased tissue or to assist the differentiation of stem cells or other cell types. For instance, a tumor could be “salted” with tryptophan to eliminate the trypophan sink and encourage T-cells to proliferate, or to aid the T cells in locating it. Alternately, a tumor could be surrounded with a region of cytokine that prevents cell motility, in order to prevent metastasis. Alternately, cells could be used as cytokine beacons. If cells are used as cytokine beacons, they could be encased in a Feridex-alginate magnetocapsule that would protect them from other immune cells and make them visible on MRI, while still allowing them to secrete cytokines. Alternately, morphogens could be used for wound repair by either stimulating cell growth and proliferation, or by stimulating stem cell differentiation.

In another aspect of the invention, the progress of therapy may be monitored and the medical history of patients determined using one or more elements, markers, ligands, or other characteristics of the immune system. While traditional medical history is based on paper or electronic records, or a questionnaire completed by the patient, a complete medical history of the patient's previous diseases can be determined from the memory cells of the immune system.

In one embodiment, a patient's medical history may be determined by sampling memory cells against the above discussed antigen and address assays to determine what diseases the patient has had and where. Alternately, similar methods could be used to monitor the patient during and after therapy to insure that the immune system is no longer detecting specific antigens. By monitoring cytokines and dendritic cells and other aspects of the immune system the progress in eliminating the disease can be determined.

Having described several aspects of this invention, it should be appreciated that various alterations, modifications and improvements will occur to those of skill in the art. Such alterations, modifications and improvements are intended to be part of this disclosure and are intended to be within the spirit and scope of the invention. Thus, the description and drawings herein are intended to be illustrative, not limiting.

1. A method for determining disease location in a body, comprising:
analyzing one or more elements, markers, ligands, or other characteristics of an immune system of a body; and
determining a location in the body at which a disease is present based on a result of analyzing the one or more characteristics of the immune system.

2. The method of claim 1, wherein the step of analyzing comprises:
analyzing one or more of cells, cytokines, antibodies, soluble factors, or gradients related to the immune system.

3. The method of claim 2, wherein the step of analyzing comprises:
analyzing cells using one or more of the following:
assaying a surface of the cells,
extracting cell contents,
imaging the cells,
assaying factors secreted by the cells,
tracking a migratory behavior of the cells, or
assaying one or more binding characteristics of the cells.

4. The method of claim 3, wherein the step of analyzing cells comprises:
assaying a surface of the cells by assaying one or more of T-cell receptors, MHC-antigen complexes, integrins, selectin ligands, chemokine receptors, B-cell receptors, KIR receptors, cytokine receptors, or NKG2 receptors.

5. The method of claim 3, wherein the step of analyzing cells comprises:
assaying factors secreted by the cells, wherein the factors include cytokines that recruit other cells.

6. The method of claim 3, wherein the step of analyzing cells comprises:
assaying factors secreted by the cells, wherein the factors include factors that imprint tissue-specific homing properties onto other cells of the immune system.

7. The method of claim 1, wherein the step of analyzing comprises:
analyzing characteristics of the immune system including body temperature or other physical characteristics of the body with a spatial resolution of about 500 micrometers or greater.

8. A method for detecting failure of one or more phases of an immune system, comprising:
analyzing one or more characteristics of the immune system of a body during a response of the immune system to a cancer in the body; and
determining failure of at least one phase of the immune systein in responding to the cancer based on a result of analyzing the one or more characteristics of the immune system.

9. The method of claim 8, wherein the step of analyzing comprises:
assessing a function of the immune system at one or more phases of its sequential process; and
wherein immune system failure is determined based on a result of assessing the function of the immune system at one or more phases of its sequential process in responding to the cancer.

10. The method of claim 9, wherein the step of analyzing comprises:
assessing the function of the immune system at multiple phases of its sequential process.

11. The method of claim 9, wherein phases of the sequential process include activation of the innate immune system and activation of the adaptive immune system.

12. The method of claim 9, wherein phases of the sequential process include acute phase response, behavior of macrophages, natural killer cell response, regulatory T cell behavior, interaction of cells via integrins, recruitment and activation of antigen presenting cells, migration of antigen presenting cells, activation of T cells, tissue specific homing of T-cells, and disease neutralization activity of T cells.

13. The method of claim 9, wherein the step of assessing comprises:
assessing a function of the natural killer cell response phase of the sequential process.

14. The method of claim 13, wherein immune system failure is determined in response to determining a failure of natural killer cells to develop a cytotoxic reaction to diseased cells, or a failure of natural killer cells to avoid developing a cytotoxic reaction to healthy cells.

15. The method of claim 14, wherein the step of assessing comprises:
assessing natural killer cells by determining a number of cells present, and/or determining characteristics of natural killer cell receptors and/or the ligands of the receptors.

16. The method of claim 15, wherein the natural killer cell receptors include activating, inhibitory, and/or binding receptors, and characteristics of the receptors and/or ligands of the receptors include phenotype, expression level, concentration, and/or binding affinity.

17. The method of claim 16, wherein activating, inhibitory, and binding receptors on natural killer cells include KIR, NKG2, Fc, cytokine receptors, and integrins.

18. The method of claim 16, wherein the ligands of activating, inhibitory, and binding receptors include cytokines, HLA molecules, the Fc ends of antibodies, and integrin ligands.

19. The method of claim 8, further comprising:
predicting disease outcome with respect to the cancer based on a result of determining a failure of a phase of the immune system.

20. The method of claim 8, further comprising:
classifying, categorizing, or describing a disease based on a result of determining a failure of a phase of the immune system.

21. A method for developing and/or implementing a treatment for disease, comprising:
analyzing one or more elements, markers, ligands, or other characteristics of an immune system of a body;
determining a location of the body at which a disease is present and/or immune system failure based on a result of analyzing the one or more characteristics of the immune system; and
determining a treatment for a disease based on information about disease location and/or immune system failure.

22. The method of claim 21, wherein the step of determining a location and/or immune system failure comprises:
determining information about disease location, and
wherein the step of determining a treatment comprises;
using information about disease location to target or deliver a therapy to specific cells or tissues.

23. The method of claim 22, wherein the step of using information about disease location includes delivering a therapy to specific cells or tissues using one or more properties of the immune system that enables immunologic cells to home to specific tissues.

24. The method of claim 21, wherein the step of determining a location and/or immune system failure comprises:
determining information about immune system failure, and
wherein the step of determining a treatment comprises;
using information about immune system failure to devise a treatment that specifically corrects the immune system failure, and enables the immune system to neutralize the disease.

25. The method of claim 24, wherein the treatment that specifically corrects the immune system failure includes an addition or removal of cells, cytokines, hormones, antibodies, proteins, or other molecules with respect to the body.

26. The method of claim 24, wherein the treatment that specifically corrects the immune system failure includes an addition or removal of ligands for activating or inhibitory receptors on immune cells with respect to the body, and/or by blocking of activating or inhibitory receptors on immune cells in the body.

27. The method of claim 24, wherein the disease is cancer and the immune system failure is corrected by promoting a likelihood of a cytotoxic reaction to cancer cells.

28. The method of claim 27, wherein the cytotoxic reaction is promoted by a therapy that increases signaling of activating receptors and/or reduces signaling of inhibitory receptors on natural killer cells.
